(19) United States
(12) Patent Application Publication
(10) Pub. No.: US 2010/0322866 A1
(43) Pub. Date: Dec. 23, 2010

(54) DEVICE, SYSTEM AND METHOD FOR IN-VIVO ANALYSIS

(76) Inventor: Elisha Rabinovitz, Haifa (IL)

Correspondence Address: Pearl Cohen Zedek Latzer, LLP, 1500 Broadway, 12th Floor, New York, NY, 10036, US

(21) Appl. No.: 12/294,679

(22) PCT Filed: Apr. 10, 2007
(86) PCT No.: PCT/IL07/00457

§ 371 Date: Jul. 28, 2009

Related U.S. Application Data
(60) Provisional application No. 60/788,116, filed on Apr. 3, 2006.

(51) Int. Cl. A61K 49/00; G01N 21/00

(52) U.S. Cl. 424/9.6; 424/9.1; 436/164

(57) ABSTRACT

A method for detecting the presence of a substance such as an antigen, the method including the steps of: maintaining a signaling material at a first pH within a liposome; contacting the liposome with a sample having a second pH such that if the substance is present in the sample the liposome will rupture thereby exposing the signaling material to the second pH; and detecting a change in an optical property, such as color, of the signaling material, the change caused by the exposure to the second pH.

### FIELD OF THE INVENTION

The present invention relates to point of care analysis in general and specifically to in vivo body fluids analysis utilizing liposomes and/or nano-containers.

### BACKGROUND OF THE INVENTION

In some cases diseases, such as cancer, can be detected by analyzing the blood for tumor specific markers, typically, specific antibodies. In vitro testing of body fluid samples for the presence of a suspected substance is common. For example, immunoassays are widely used for the determination, either qualitative or, quantitative, of a variety of substances, such as peptides, proteins, enzymes, hormones, vitamins, drugs, carbohydrates, etc. Typically, these in vitro methods of detection are carried out in the lab using cumbersome and complicated automatic machines due to the complicated requirements of such procedures which may involve a plurality of reactions and perhaps different conditions for each reaction.

In some cases the appearance of antibodies in the blood stream may occur at a late stage of the disease. Also, these methods of detection, when carried out in vitro, may not easily enable the localization or identification of the origin of an abnormally occurring substance. In many instances localizing an abnormally occurring substance in a body lumen greatly contributes to the identification of pathology. For example, the presence of elevated concentrations of red blood cells in the gastrointestinal (GI) tract may indicate different pathologies, depending on the location of the bleeding along the GI tract and thus may contribute to the facile treatment of the identified pathology.

Devices and systems for in-vivo imaging may be used, for example, to acquire in vivo images of the GI tract however, imaging alone may typically not enable early detection of pathology, such as cancer.

### SUMMARY OF THE INVENTION

Embodiments of the present invention include the use of polymerosomes or nano-containers in detecting pathologies. Some embodiments may be designed for use ex vivo or in vitro while other embodiments of the invention may allow, for example, in-vivo analysis or in-vivo analysis and imaging.

According to one embodiment a nano-container may contain a marker and may include a targeting element.

A marker, which may be an indicator, a signaling agent or a signaling material, may typically include an optically visible substance, such as a dye. A targeting element may typically promote orientation of targeted molecules or compounds in the environment to the nano-container. According to an embodiment of the invention a targeting element may include, for example, an antibody or antibody hotspot, a sugar, a peptide, a protein, an oligonucleotide, a lipid, a neuromediator, a hormone, a vitamin, or derivatives thereof. According to some embodiments of the invention reaction of a targeting element included in the nano-container with a targeted molecule or compound in the environment may bring about a conformational transformation of the nano-container, which may induce degradation, perforation, rupture or other openings in the nano-container, thus releasing the marker from the nano-container. Thus, according to one embodiment, the marker contained in the nano-container will become visible only upon reaction of the targeting element with a targeted molecule.

According to some embodiments of the invention the marker may be sensitive to changes in its environment and may display different optical properties depending on the environment. According to other embodiments a marker need not be detected by its optical properties. According to some embodiments a marker may be detected by properties such as electric charge, conformational changes, weight etc.

Embodiments of the invention provide a method for pathology detection. According to one embodiment nano-containers containing a marker and a targeting element are introduced into a patient's body. An in-vivo imaging device is inserted into the patient's body, for example, a swallowable imaging capsule is inserted to the GI tract. If a targeted molecule or determinant are present in the patient's body environment, the nano-container will be attracted to them and upon reacting with the targeted molecule or determinant the nano-container may erupt or otherwise open to enable the marker contained in the nano-container to be imaged by the in-vivo imaging device.

According to another embodiment an in vitro test may be provided. According to one embodiment a sample taken from a patient may be mixed in vitro with nano-containers according to embodiments of the invention. A change in optical properties of the sample may indicate the presence of a targeted molecule in the sample.

A kit according to an embodiment of the invention may be used as a “point of care”, enabling a care giver to perform analysis of body samples or other samples on the spot, not having to use, for example, the services of a special laboratory.

Embodiments of the invention provide a system for pathology detection. The system may include an in-vivo device that is an autonomous swallowable capsule, which may or may not include an imager and a transmitter to transmit data to an external receiver and monitor.

Embodiments of the invention may provide additional and/or other benefits and/or advantages.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a nano-container in accordance with an embodiment of the invention;

FIG. 2 is a schematic illustration of an in-vivo sensing system in accordance with some embodiments of the invention;

FIG. 3 is a schematic illustration of an in-vivo imaging device in accordance with an embodiment of the invention;

FIG. 4 is a schematic illustration of an in-vivo imaging device in accordance with another embodiment of the invention;

FIG. 5 is a schematic illustration of an in-vivo imaging device in accordance with yet another embodiment of the invention;

FIG. 6 is a schematic illustration of an in-vivo analysis system in accordance with some embodiments of the invention;

FIG. 7 is a schematic illustration of an analysis device in accordance with some embodiments of the invention; and

FIG. 8 is a flowchart of a method of in-vivo analysis in accordance with some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Some embodiments of the present invention are directed to a one time use in vitro detection and/or analysis device.

Other embodiments are directed to a swallowable in-vivo device that passively or actively progresses through a body lumen, e.g., the gastro-intestinal (GI) tract, typically, pushed along by natural peristalsis. According to an embodiment of the invention the in-vivo device may include a reaction chamber and a transmitter. The reaction chamber, into which a sample may be gathered in vivo, may include nano-containers according to embodiments of the invention. Changes occurring in the reaction chamber due to the presence of a targeted molecule in the sample may be transmitted by the transmitter to an external receiving system. According to an embodiment of the invention the transmitter is a wireless transmitter, for example, an RF transmitter. According to some embodiments the in vivo device may include a light source and an imaging system (which may include, for example, an optical system and an imager) for imaging a reaction occurring in the reaction chamber. According to some embodiments the in vivo device may include a power source for powering electrical components of the device.

Embodiments of the invention may include a receiving system to receive data transmitted from the in vivo device and a display to display the transmitted data. Embodiments of the invention may include a processor to process the data prior to displaying it.

Embodiments of the invention are directed to a swallowable in-vivo device in which essentially includes an image sensor or an imager. Other sensors may be included, for example, a pH sensor, a temperature sensor, a pressure sensor, sensors of other in-vivo parameters, sensors of various in-vivo substances or compounds, or the like.

Devices, systems and methods according to some embodiments of the present invention, including for example in-vivo sensing devices, receiving systems and/or display systems, may be similar to embodiments described in U.S. Pat. No. 5,604,531 to Iddan et al., entitled “In-vivo Video Camera System”, and/or in U.S. patent application Ser. No. 09/800,470, entitled “A Device and System for In-Vivo Imaging”, filed on Mar. 8, 2001, published on Nov. 1, 2001 as United States Patent Application Publication Number 2001/0035902, and/or in U.S. patent application Ser. No. 10/046,541, entitled “System and Method for Wide Field Imaging of Body Lumens”, filed on Jan. 16, 2002, published on Aug. 15, 2002 as United States Patent Application Publication Number 2002/0109774, and/or in U.S. patent application Ser. No. 10/046,540, entitled “System and Method for Determining In-vivo Body Lumen Conditions”, filed on Jan. 16, 2002, published on Aug. 15, 2002 as United States Patent Application Publication Number 2002/0111544, all of which are hereby incorporated by reference in their entirety. Devices and systems as described herein may have other configurations and/or sets of components. For example, an external receiver/recorder unit, a processor and a monitor, e.g., in a workstation, such as those described in the above publications, may be suitable for use with some embodiments of the present invention. Devices and systems as described herein may have other configurations and/or other sets of components. For example, the present invention may be practiced using an endoscope, needle, stent, catheter, etc. Some in-vivo devices may be capsule shaped, or may have other shapes, for example, a peanut shape or tubular, spherical, conical, or other suitable shapes.

Embodiments of the present invention include and are directed to, a swallowable in-vivo device passing through the GI tract. In some embodiments, the in-vivo device may optionally include a sensor other than an imager, and/or other suitable components.

Embodiments of the in-vivo device are autonomous and self-contained. For example, the in-vivo device includes a capsule or other unit where all the components are substantially contained within a container, housing or shell, and where the in-vivo device does not require any wires or cables to, for example, receive power or transmit information. The in-vivo device may communicate with an external receiving and display system to provide display of data, control, or other functions. For example, power is provided by an internal battery or an internal power source. Other embodiments may have other configurations and capabilities. For example, components may be distributed over multiple sites or units.

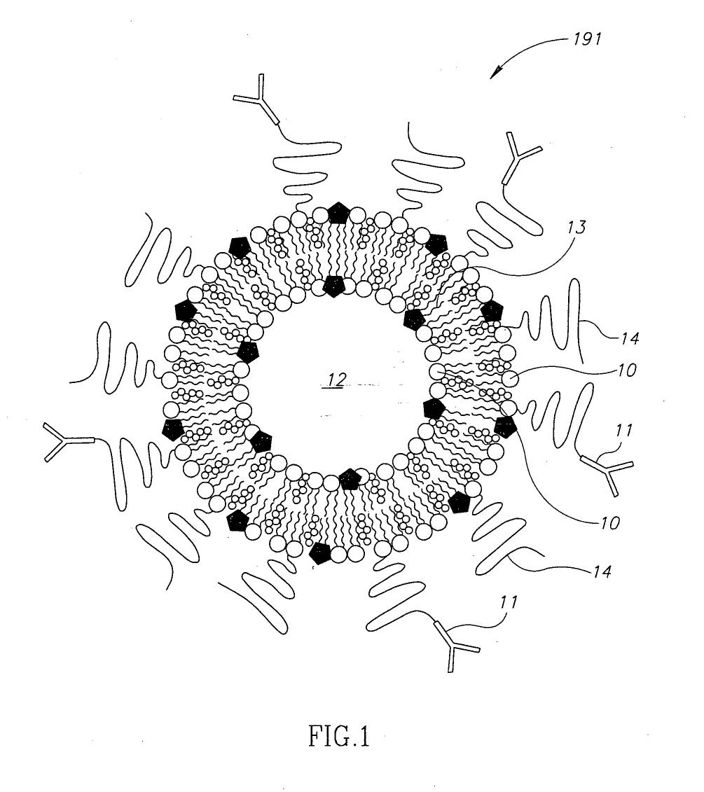

Reference is now made to FIG. 1 which schematically describes a nano-container or liquid crystal polymer designed for release (in vivo and/or in vitro) of entrapped marker material. According to one embodiment the nano-container is designed to release the marker in a controlled manner, for example, in discrete pulses as a result of specific stimuli such as exposure of the nano-container to a targeted agent. The nano-container (e.g., polymerosome or liposome) 191 can be made of natural or synthetic vesicle-forming lipids 10. Lipids, being bipolar molecules, having a hydrophilic head and hydrophobic tail, naturally form a circular bilayer surrounding water compartment which, when immersed in water, arranges itself so that the hydrophilic regions of the lipid point toward the water and the hydrophobic regions of the lipid point away from the water. This forms two separate water interacting surfaces. Substances such as drugs or dyes can be dissolved in different compartments (e.g., 12 and 13) of such a liposome. Lipids 10 used to create the nano-container 191 may include phospholipids such as sphingomyelin or phosphatidylcholine, steroids such as cholesterol, ceramides, sphingolipids and other polymers either synthetic or natural. For example, nano-containers can include various carbohydrate polymers having hydrophilic/hydrophobic structures, such as glycoproteins, glycolipids, viral proteins, polysaccharides, lipo-polysaccharides and other oligosaccharides. Other molecules can be used as known in the art. Polymers such as polyethylene glycol (PEG) and other surfactants can also be used, typically to form a protective layer 14 on hollowed nano-structures, typically termed polymerosomes. Such lipid-made containers having a shape of a ball within a ball and having an inner diameter smaller than about 1 micron may be called nano-containers. In some embodiments nano-containers 191 can be trapped in a larger container creating a multi-layered nano-container. Typically, multiple layered liposomes are easy to produce but it is more difficult to release materials that are entrapped in the inner layers of these liposomes. Different solutions can be preferred for different applications e.g. for nano-containers designed to penetrate narrow blood vessels bi-layered liposomes may be preferred. For typically stable profiles double diffusion barrier containers may be preferred. According to some embodiments the release of an entrapped substance for example from cavities 12 or 13 into the surrounding environment is governed by the permeability of both the liposome and additional layers (such as protective layer 14). Permeability of a liposome may be enhanced by including an enzyme such as phospholipase within some or all of the liposomes or the nanoparticles.

Protective layer 14 may be used in the coupling of a ligand 11 with well defined affinity for a specific targeted agent. Ligand 11 may include an antigen, receptor, cell or expression on cell, for example anti-target monoclonal antibodies, or an active part of an antibody, a “hotspot” of an antibody or a protein (e.g., minimal functional domains involved in protein-to-protein interactions and sufficient to induce a biological or chemical response) glycoproteins, glycolipids or “glycocalyx” or bacterial poly-saccharides that can either covalently or non-covalently be attached to specific receptors or antigen or to a antigenic moiety expressed on target sites (e.g., GI tract tissue). Ligand 11 may be attached to nano-container 191 in other ways. Ligand 11 may act to confer specificity of the nano-container to targeted molecules and to initiate a change of permeability in the nano-container. According to one embodiment ligand 11 is anchored to the nano-container such that once it reacts with a targeted molecule or agent it will cause a chain of events at the end of which the surface of liposomes will be perforated for example by a rupture or disintegration of nano-container 191. Such events may include direct perforation of the nano-container or indirect activation for example by release of enzyme like phospholipase that will in turn disrupt the liposome. At the end of the process the content encapsulated in volume 12 or 13 is exposed or otherwise released.

In some embodiments, volume 12 and/or 13 of the nano-containers 191 may be filled with, for example, a solution containing water soluble hydrophilic signaling material. Such signaling material may be, for example, a pH-sensitive substance or indicator, a fluorescent substance or indicator, or another substance or indicator which may modify its optical properties (e.g., its color or its light emission or absorption properties) upon modification of the signaling material environment or upon reaction between the signaling material and another substance (e.g., targeted protein or an analyte present in body fluid). For example, in some embodiments, signaling material may initially (e.g., when stored within liposome or nano-container 191) have a first color, e.g., blue; and may modify its color to a second, different color, e.g., yellow, according to the prevalent pH or other environmental parameters (e.g., temperature, enzymatic environment, bacterial or viral environment, electric charge, etc.). According to an embodiment of the invention volume 13 and/or 12 may include or may be filled with water insoluble or hydrophobic or lipophilic signaling material which will modify its optical properties (e.g., its color or its light emission or absorption properties) upon modification of the signaling material environment or upon reaction between the signaling material and another substance (e.g., targeted protein or an analyte present in body fluid). For example, the signaling material or marker may include a solution containing macromolecule with fluorescent or other dye properties, capable of changing optical properties in a pH dependant manner. This marker, while in the nano-container can be kept in a pH that is not typical to an in vivo environment and thus display a first color, whereas, upon being released from the nano-container into a different pH can display a second color. In some embodiments, for example, in a multiple layer nano-container different zones may be used for different solutions. Other numbers or locations of layers, shells, zones and/or nano-compartments may be used.

Upon rupturing of the liposomes or nano-containers 191 and exposure of, for example, a pH sensitive color to the sample, a change of color may occur that may be optically detectible, for example, by an in vivo imaging device and/or visible, for example to a human eye.

In some embodiments test tubes with for example different solutions containing variety of analyte specific nano-containers 191 or any combination of those can be prepared. Samples, such as body fluid samples, food samples, tissue samples or other suitable samples may be added to the test tube. Nano-container 191 may contain for example a pH sensitive indicator that changes color when it is exposed to a different pH than the liposome internal pH and its outer layer may include an antibody to for example a certain determinant (e.g., of a tumor, bacteria or virus). The antibody which coats or is attached or anchored to the liposome outer shell will react with the determinant if it is present in the sample. Reaction with the determinant will cause a change in the nano-container permeability which will cause the pH sensitive indicator to be exposed to the pH of the sample (which is typically different than the liposome internal pH). The exposed indicator will thus change color. The change of color (or any other optical property) may be detected by eye or by imaging or other light detecting devices.

According to some embodiments a marker may go through a change that is not optical (such as a change in conformation, charge etc.) and this change may be detected by suitable measuring devices.

A system according to some embodiments may be used for example for field analysis of water suspected as contaminated, and in another embodiment it can be used for example to determine the presence of a virus, bacteria or a biomarker in blood or plasma or other body fluids such as urine.

The following non limiting examples are described to illustrate embodiments of the invention.

### Example 1

Trypsin inhibitor is a known biomarker for acute renal failure. Typsin is an enzyme known to cleave proteins containing the basic amino acid lysine. According to one embodiment of the present invention, a diagnostic kit can be prepared. The diagnostic kit, according to one embodiment, may include

A: liposomes based on phospholipids containing lysine or a mixture of polymersomes with proteins containing lysine in a solution with a pH of about 3.5 or lower;

B: pH indicator, for example bromocresol green having a yellow color in a pH lower than 3.8 and a blue color in a pH higher than 5.5. Other suitable indicators can be used. The indicator is entrapped in the liposomes;

C: Trypsin, which is typically inactive at a low pH, may be mixed with the liposome or attached to polysaccharides coating the liposome using technologies well known in the art. The above may be mixed and may be lyophilized and introduced into a test tube containing a urine sample (the sample can be buffered to pH 8 to accelerate the process). At this stage the liquid in the test tube is yellow due to the acidic bromocresol green indicator that is sealed inside the liposomes. As a result of the high pH in the sample the trypsin will restore its activity and start to cleavage the lysine containing liposome. The solution with bromocresol green in its acidic yellow stage will be exposed to the high pH of the urine outside the liposome and change its color to blue. However, if the urine has a high concentration of trypsin inhibitor the cleavage of the lysine containing liposome will be prevented or at least delayed (compare to urine that does not contain the inhibitor) indicating possible renal failure.

### Example 2

Secretory Phospholipase A2 (sPLA2) is an enzyme that is active in the intercellular space. It is over expressed in inflammatory as well as cancerous tissues. The diagnostic kit, according to one embodiment, may include

A: liposomes based on phospholipids containing for example a fatty acid with an acryl group at 2-lysophospholipid or similar phospholipase that can be hydrolyzed by ePLA2, The liposomes are prepared in a solution with a pH of about 3.5 or lower;

B: pH indicator for example Chlorophenol red having a yellow color in a pH lower than 5.2 and a purple color at a pH higher than 6.8. Chlorophenol is nontoxic. The indicator is entrapped in the liposomes;

C: the liposome is conjugated with a specific antibody to the specific inflammation for example IBD in the colon or cancerous polyp.

In one embodiment the liposomes are encapsulated in a pill and are released only when the colon is reached e.g. liposomes are contained in a pill that is coated with a pH sensitive coating that is degraded at pH>7.

Once the pill is administered to a subject and once it reaches the colon the pill will decompose and release the liposomes. Since polyps (even benign) are perforated tissue some of the liposomes may for example be concentrated in polyps staining them in yellow. A high concentration of liposomes will be attached to the cancerous tumor or the inflammation with the targeted antigen. In those cases the ePLA2 will hydrolyze the liposome releasing its content. As the pH of the colon is >7 the color of the indicator will change from yellow to purple thus differentiating between benign and cancerous polyps.

Capsule endoscopy or standard endoscope may be used to detect the stained tumors

### Example 3

Liposomes are prepared as described in Example 2 except that the signaling agent is an IR sensitive material. Said liposomes are suspended in physiological solution and introduced into the systemic blood stream by injection by a syringe or by intravenous (IV) injection or any other suitable delivery mechanisms as known in the art.

Once the liposomes are released they spread, in the body but specifically accumulate in tumors enriched with the targeted antigen. An in-vivo imaging device such as an imaging capsule or endoscope equipped with an IR source may be inserted into the patient's body, optionally after waiting a pre-defined period to allow the liposomes or nano-containers to spread and reach the tumor. The IR light may penetrate the tissue and excite the IR signaling material even if the tumor is sub facial. The light emitted by the excited IR signaling material can thus be detected by the imager of the capsule or the endoscope enabling the detection of deep tissues tumors.

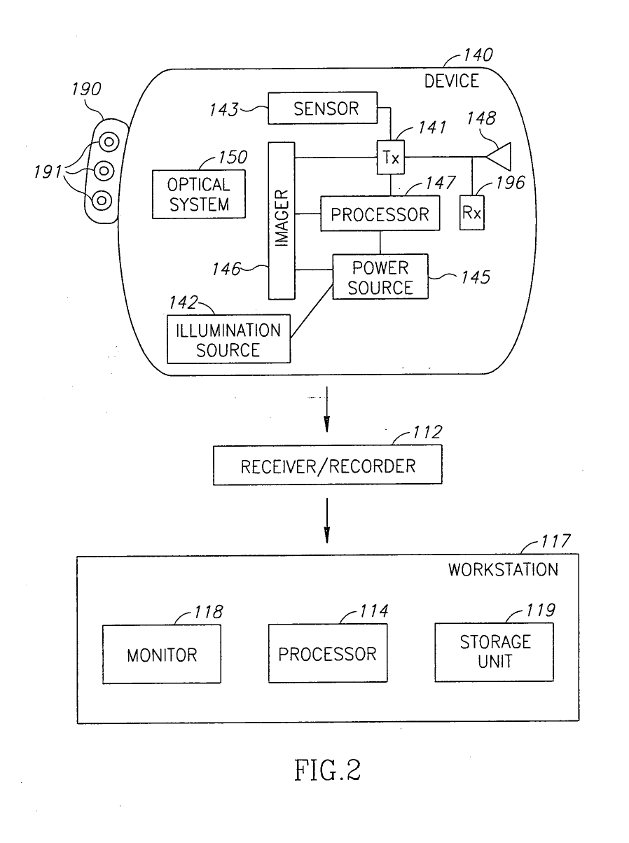

FIG. 2 is a schematic illustration of an in-vivo sensing system 100 in accordance with some embodiments of the invention. One or more components of system 100 may be used in conjunction with, or may be operatively associated with, the devices and/or components described herein or other in-vivo devices in accordance with embodiments of the invention. Device 140 essentially includes an imager 146, one or more illumination sources 142, a power source 145, and a transmitter 141. Device 140 is implemented using a swallowable capsule.

Device 140 includes an autonomous swallowable capsule, but device 140 may have other shapes. Embodiments of device 140 are autonomous, and are self-contained. For example, device 140 may typically be a capsule or other unit where all the components are substantially contained within a container or shell or housing, and where device 140 does not require any wires or cables to, for example, receive power and/or transmit information. In some embodiments, device 140 may be autonomous and non-remote-controllable; in another embodiment, device 140 may be partially or entirely remote-controllable.

Outside a patient's body may be, for example, an external receiver/recorder 112, which may include, or may associated with, one or more antennas (or antenna elements), optionally arranged as an antenna array. Receiver/recorder 112 may receive signals transmitted by the in-vivo device 140, for example, signals carrying image data, sensed data, control data, or the like. Receiver/recorder 112 may, for example, store the received data in a memory unit or a storage unit, or may display the information on a display unit (e.g., in real time or not in real time), for example, using hand-held device or computer.

Additionally, outside a patient's body may be, for example, a storage unit 119, a processor 114, and a monitor 118, which may optionally be implemented as a workstation 117, e.g., a computer or a computing platform. Workstation 117 may be connected to receiver/recorder 112 through a wireless or wired link or connection. Workstation 117 may receive from receiver/recorder 112 data that is received and/or recorded by receiver/recorder 112. In some embodiments, workstation 117 may receive the data from receiver/recorder 112 substantially in real-time, and/or while receiver/recorder 112 continues to receive and/or record data from the in-vivo device 140 and while the in-vivo device 140 is operational and/or in-vivo. In some embodiments, device 140 may communicate (e.g., directly or indirectly) with the external receiving and display system (e.g., workstation 117 or monitor 118) to provide display of data, control, or other functions.

Device 140 essentially includes an in-vivo video camera, for example, imager 146, which may capture and transmit images of, for example, the GI tract while device 140 passes through the GI lumen. Other lumens and/or body cavities may be imaged and/or sensed by device 140. In some embodiments, imager 146 may include, for example, a Charge Coupled Device (CCD) camera or imager, a Complementary Metal Oxide Semiconductor (CMOS) camera or imager, any other solid state camera or imager, a light detector, a linear imaging sensor, a line imaging sensor, a full frame imaging sensor, a “camera on chip” imaging sensor, a digital camera, a stills camera, a video camera, or other suitable imagers, cameras, or image acquisition components.

Transmitter 141 of device 140 may essentially include a wireless transmitter, e.g., able to operate using radio waves, able to transmit Radio Frequency (RF) signals, or able to transmit other types of communication signals. For example, transmitter 141 may transmit wireless signals utilizing an antenna 148. Other wireless methods of transmission may be used.

In some embodiments, device 140 may optionally include a receiver 196, for example, a wireless (e.g., RF) receiver, able to receive signals from an external transmitter. The received signals may include, for example, control signals or commands, e.g., to activate and/or otherwise control one or more components of device 140. Receiver 196 may receive signals, e.g., from outside the patient's body, for example, through antenna 148 or through a different antenna or receiving element. In some embodiments, signals or data may be received by a separate receiving unit in device 140. In some embodiments, transmitter 141 and receiver 196 may optionally be implemented using a transceiver unit or an integrated transmitter-receiver unit.

In some embodiments, imager 146 in device 140 may be operationally connected to transmitter 141. Transmitter 141 may transmit images and/or data to, for example, external transceiver or receiver/recorder 112 (e.g., through one or more antennas), which may send the data to processor 114 and/or to storage unit 119. Transmitter 141 may also include control capability, although control capability may be included in a separate component, e.g., a controller or processor 147. Transmitter 141 may include any suitable transmitter able to transmit image data, numerical data, other sensed data, and/or other data (e.g., control data) to a receiving device. Transmitter 141 may also be capable of receiving signals/commands, for example from an external transceiver. For example, in some embodiments, transmitter 141 may include an ultra low power Radio Frequency (RF) high bandwidth transmitter, possibly provided in Chip Scale Package (CSP).

In some embodiment, transmitter 141 may transmit/receive data via antenna 148. Transmitter 141 and/or another unit in device 140, e.g., a controller or processor 147, may include control capability, for example, one or more control modules, processing modules, circuitry and/or functionality for controlling device 140, for controlling the operational mode or settings of device 140, and/or for performing control operations or processing operations within device 140.

Power source 145 may essentially include one or more batteries or power cells. For example, power source 145 may include silver oxide batteries, lithium batteries, other suitable electrochemical cells having a high energy density, or the like. Other suitable power sources may be used.

Power source 145 is to be internal to device 140, and/or does not require coupling to an external power source, e.g., to receive power. Power source 145 may provide power to one or more components of device 140, for example, continuously, substantially continuously, or in a non-discrete manner or timing, or in a periodic manner, an intermittent manner, or an otherwise non-continuous manner. In some embodiments, power source 145 may provide power to one or more components of device 140, for example, not necessarily upon-demand, or not necessarily upon a triggering event or an external activation or external excitement. Optionally, in some embodiments, transmitter 141 may include a processing unit or processor or controller (e.g., controller or processor 147), for example, to process signals and/or data generated by imager 146. In some embodiment, the processing unit may be an independent unit or integrated with another component within device 140, e.g., controller or processor 147, or may be implemented as an integral part of imager 146, transmitter 141, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In some embodiments, for example, the processing unit or controller may be embedded in or integrated with transmitter 141, and may be implemented, for example, using an ASIC.

In some embodiments, imager 146 may acquire in-vivo images, for example, continuously, substantially continuously, or in a non-discrete manner, for example, not necessarily upon-demand, or not necessarily upon a triggering event or an external activation or external excitement; or in a periodic manner, an intermittent manner, or an otherwise non-continuous manner.

In some embodiments, transmitter 141 may transmit image data continuously, or substantially continuously, for example, not necessarily upon-demand, or not necessarily upon a triggering event or an external activation or external excitement; or in a periodic manner, an intermittent manner, or an otherwise non-continuous manner.

In some embodiments transmitter 141 may transmit data other than image data.

Device 140 may essentially include one or more illumination sources 142, for example one or more Light Emitting Diodes (LEDs), “white LEDs”, monochromatic LEDs, Organic LEDs (O-LEDs), thin-film LEDs, single-color LED(s), multi-color LED(s), LED(s) emitting viewable light, LED(s) emitting non-viewable light, LED(s) emitting Infra Red (IR) light, an emissive electroluminescent layer or component, Organic Electro-Luminescence (OEL) layer or component, or other suitable light sources.

Illumination sources 142 may, for example, illuminate a body lumen or cavity being imaged and/or sensed. Device 140 may optionally include an optical system 150, for example, one or more optical elements, lenses, composite lens assemblies, magnifying lens, optical filters, prisms, gratings, plane mirrors, curved mirrors, concave mirrors or elements, convex mirrors or elements, reflective surfaces, reflective elements, light tunnels, light diverting elements, light focusing elements, or any other suitable optical elements. Optical system 150 may, for example, aid in focusing reflected light onto imager 146, focusing illuminated light, and/or performing other light processing operations.

In some embodiments, illumination source(s) 142 may illuminate continuously, or substantially continuously, for example, not necessarily upon-demand, or not necessarily upon a triggering event or an external activation or external excitement. In some embodiments, for example, illumination source(s) 142 may illuminate a pre-defined number of times per second (e.g., two or four times), substantially continuously, e.g., for a time period of two hours, four hours, eight hours, or the like; or in a periodic manner, an intermittent manner, or an otherwise non-continuous manner.

Components of device 140 are to be enclosed within a housing or shell, e.g., capsule-shaped, oblong, oval, spherical, tubular, peanut-shaped, or having other suitable shapes and/or dimensions. The housing or shell may be substantially transparent or semi-transparent, and/or may include one or more portions, windows or domes (e.g., a dome-shaped window, or multiple dome-shaped windows) which may be substantially transparent or semi-transparent. For example, one or more illumination source(s) 142 within device 140 may illuminate a body lumen through a transparent or semi-transparent portion, window or dome; and light reflected from the body lumen may enter the device 140, for example, through the same transparent or semi-transparent portion, window or dome (e.g., the window on dome on which liposomes or nano-containers 191 may be located) or, optionally, through another transparent or semi-transparent portion, window or dome, and may be received by optical system 150 and/or imager 146. In some embodiments, for example, optical system 150 and/or imager 146 may receive light, reflected from a body lumen, through the same window or dome through which illumination source(s) 142 illuminate the body lumen.

Workstation 117 may include data processor 114 able to analyze the data received from device 140, and optionally able to separate images related to imaging the body lumen from images or data related to molecular analysis by the liposomes or nano-particles 191. Data processor 114 may be in communication with storage unit 119, e.g., able to transfer frame data to and/or from storage unit 119. Data processor 114 may provide the analyzed data to monitor 118, where a user (e.g., a physician) may view or otherwise use the presented data. Data processor 114 may analyze the data received via external receiver/recorder 112 or (e.g., directly) from device 140, and may be in communication with storage unit 119, e.g., transferring frame data to and from storage unit 119. Data processor 114 may provide the analyzed data to monitor 118, where a user (e.g., a physician) may view or otherwise use the data. In some embodiments, data processor 114 and/or workstation 117 may be configured for real time processing, and/or may be implemented using a hand-held device. In another embodiment, post processing may be performed, and data or images may be viewed at a later time (e.g., not in real time). In the case that control capability (e.g., delay, timing, etc) is external to device 140, a suitable external device (such as, for example, data processor 114 or external receiver/recorder 112 having a transmitter or transceiver) may transmit one or more control signals to device 140.

Monitor 118 may include, for example, one or more screens, monitors, or suitable display units. Monitor 118, for example, may display one or more images or a stream of images captured and/or transmitted by device 140, e.g., images of the GI tract or of other imaged body lumen or cavity. Additionally or alternatively, monitor 118 may display, for example, control data, location or position data (e.g., data describing or indicating the location or the relative location of device 140), orientation data, and various other suitable data. In some embodiments, for example, both an image and its position (e.g., relative to the body lumen being imaged) or location may be presented using monitor 118 and/or may be stored using storage unit 119. Other systems and methods of storing and/or displaying collected image data and/or other data may be used.

Typically, device 140 may transmit image information in discrete portions. Each portion may typically correspond to an image or a frame; other suitable transmission methods may be used. For example, in some embodiments, device 140 may capture and/or acquire an image once every half second, and may transmit the image data to external receiver/recorder 112. Other constant and/or variable capture rates and/or transmission rates may be used.

Typically, the image data recorded and transmitted may include digital color image data; in alternate embodiments, other image formats (e.g., black and white image data) may be used. In some embodiments, each frame of image data may include 256 rows, each row may include 256 pixels, and each pixel may include data for color and brightness according to known methods. According to other embodiments a 320×320 pixel imager may be used. Pixel size may be, for example, between 5 to 6 microns; other suitable sizes may be used. According to some embodiments, pixels may be each fitted with a micro lens. For example, a Bayer color filter may be applied. Other suitable data formats may be used, and other suitable numbers or types of rows, columns, arrays, pixels, sub-pixels, boxes, super-pixels and/or colors may be used.

Optionally, device 140 may include one or more sensors 143, instead of or in addition to a sensor such as imager 146. Sensor 143 may, for example, sense, detect, determine and/or measure one or more values of properties or characteristics of the surrounding of device 140. For example, sensor 143 may include a pH sensor, a temperature sensor, an electrical conductivity sensor, a pressure sensor, or any other known suitable in-vivo sensor.

In some embodiments, device 140 may include a carrier substance 190, e.g., a hydrogel, which may be immobilized or otherwise mounted or coated on an external portion of device 140, e.g., over a dome-shaped optical window of device 140. Carrier substance 190 may include, for example, conjugated liposomes or nano-containers 191, according to embodiments of the invention.

In some embodiments, carrier substance 190 may be made of or may include crosslinked polymeric chains, in which water or water-based solutions may be dispersed or adsorbed, for example, a hydrogel, e.g., a network of polymer chains that are water-soluble, or a colloidal gel in which water is the dispersion medium, or micelles or polymeric compounds, e.g., cellulose; other absorbent or super-absorbent natural or synthetic polymers may be used. In another embodiment, dried formulations may be used, e.g. lyophilize liposomes or polymersomes may be embedded in nitrocellulose. In other embodiments, preservatives (e.g., Thimerosal, benzyl alcohol, parabens, or the like) may be used or added.

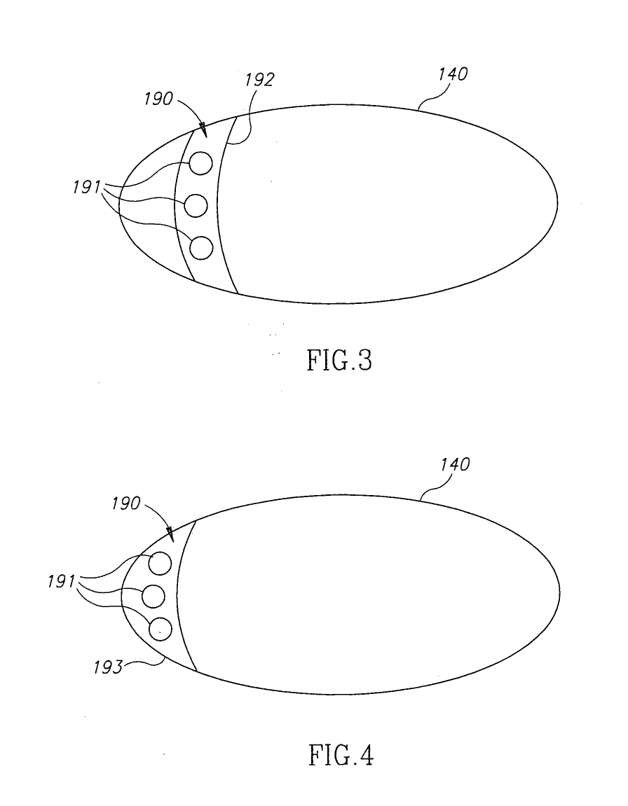

In some embodiments, for example, as illustrated in FIG. 3, carrier substance 190 and liposomes or nano-containers 191 may be placed in, or immobilized onto, a band 192, e.g., mounted or coated around or over a portion of device 140 that is in line of sight with the imager, or around or over a portion of a window or a suitable trench in the dome-shaped window of device 140.

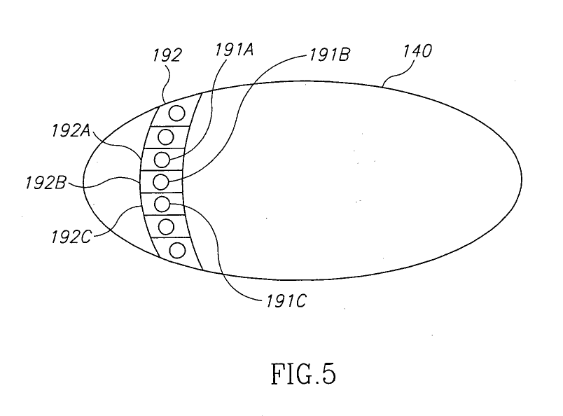

In some embodiments, for example, as illustrated in FIG. 4, carrier substance 190 and liposomes or nano-containers 191 may be placed in, or immobilized onto, an external dome-shaped portion 193, e.g., mounted or coated over a portion of device 140, or around or over a window or dome-shaped window of device 140.

In some embodiments, the carrier substance 190 (e.g., hydrogel) may be coated, for example, for preservation and/or storage while the device 140 is not in-vivo. For example, the coating may dissolve in-vivo, or only when the device 140 reaches a certain body part (e.g., the colon). In some embodiments, the coating may partially dissolve, for example, to open an inlet gate and/or an outlet gate, thereby creating a flow (e.g., a contuse flow) through the carrier substance 190.

Although FIG. 3 and FIG. 4 demonstrate the placement of carrier substance 190 and liposomes or nano-containers 191 externally to the in-vivo device 140, other suitable locations may be used. In some embodiments, for example, carrier substance 190 and liposomes or nano-containers 191 may be placed internally to device 140, e.g., within an internal compartment or chamber or channel, which may be subsequently opened in-vivo (e.g., using a dissolvable gate, a mechanical gate, or the like). Other suitable placement, mounting or coating methods may be used.

In some embodiments, conjugated liposomes or nano-containers 191 (e.g., lyophilized conjugated liposomes, or a liposome having a conjugated antibody integrated therein) may be filled with, for example, pH sensitive dye in low strength buffer possessing a pH that is not typical to an in vivo environment and thus the dye displays a first color, whereas upon being released from the nano-container to a different pH the dye will display a second color. Rupture of the liposomes or nano-containers 191 may occur as a result of a reaction with a target analyte. Upon rupturing of the liposomes or nano-containers 191 and exposure of, for example, the pH sensitive color to the sample, a change of color may occur that may be, for example, optically detectible and/or visible and/or may be imaged. In other embodiments, liposomes or nano-containers 191, e.g., polymersome, may include or may be filled with an alternate or additional molecule (e.g., fluorescence material, material having fluorescence properties, or the like) capable of changing optical properties of a substrate.

Referring again to FIG. 2, the device 140 may be inserted in-vivo by swallowing, and may pass through a body lumen. The body lumen may be sampled into the carrier 190 (either internal or externally to the device) and the liposome or nano-container 191 may be in contact with the body sample. If the body sample contains a targeted agent, such as a certain antigen of pathology, tumor cells, an infection, polyp cells, or the like a reaction will occur between the nano-container 191 and the targeted agent. The reaction may cause breaking, opening, rupture, collapsing, dissolution, fusion, or puncturing of liposome or nano-container 191, or of one or more layers (e.g., an external layer, or a portion thereof) of liposome or nano-container 191. For example, one or more holes, punctures or openings may be created, allowing the signaling material stored within liposome or nano-container 191 to exit and/or to be in contact with the body substance or with the carrier substance 190 (e.g., the hydrogel); thereby resulting in a modification of an optical property of signaling material (e.g., color or fluorescence; for example, modification from blue color to yellow color, or the like).

In some embodiments, the modification of optical property of the signaling material, or the resulting optical property of signaling material, may be imaged or otherwise sensed, e.g., by imager 146 of in-vivo device 140. For example, imager 146 may acquire one or more images, e.g., through a window or a dome-shaped window of device 140. The acquired image may include, for example, the signaling material having a modified (e.g., non-original) color, and/or a body lumen in which the reaction takes place.

Device 140 is to transmit digital color and/or black and white image information which may include color information of the liposome or nano-container 191, e.g., in discrete portions; for example, a discrete portion may correspond to an image or a frame; other suitable transmission methods may be used. In some embodiments, device 140 may capture and/or acquire an image, for example, once every half second; other capture rates, constant or variable, may be used. In some embodiments, device 140 may be used for locating the disorder or pathology, and/or for determining its nature, e.g., distinguishing between a benign and malignant polyps or tumors.

In other embodiments, for example, the liposome or nano-container 191 may be initially filled with fluorescent substance. The liposome or nano-container 191 may rupture as a result of reaction with a target, e.g., a certain antigen. The fluorescent substance stored within the liposome or nano-container 191 may be exposed or may drain out the liposome or nano-container 191 and as a result change (e.g., increase) its excitation properties. The in-vivo imager 146 or the sensor 143 (e.g., a light sensor) may acquire the images and/or the change (e.g., increase) in signal of the fluorescent substance, and the sensed data may be transmitted by transmitter 141, e.g., in addition to or instead of the relevant image data.

In some embodiments, in-vivo device 140 may be localized, e.g., using one or more localization methods, thereby allowing to determine the location or body part in which the reaction took place, e.g., the location or body part having the antigen, pathology, tumor, cancerous tumor, infection, polyp, or the like.

FIG. 5 illustrates an in-vivo device 140 having multiple types of liposomes or nano-containers 191, in accordance with some embodiments of the invention. For example, carrier substance 190 and liposomes or nano-containers 191 may be placed in, or immobilized onto, a band 192, e.g., mounted or coated around or over a portion of device 140, or around or over a portion of a window or dome-shaped window of device 140. Band 192 may include multiple portions or areas, for example, a first portion 192A, a second portion 192B, a third portion 192C, or the like; and each portion may include, for example, a different type of liposomes or nano-containers, e.g., a first type of liposomes or nano-containers 191A, a second type of liposomes or nano-containers 191B, a third type of liposomes or nano-containers 191C, respectively. For example, the first type of liposomes or nano-containers 191A may be adapted to react to a first antigen or protein, the second type of liposomes or nano-containers 191A may be adapted to react to an enzyme or protein, the third type of liposomes or nano-containers 191A may be adapted to react to a tertian hormone or protein, or the like.

In-vivo device 140 of FIG. 5 may pass through a body lumen, e.g., the GI tract. The first type of liposomes or nano-containers 191A may be in contact with the first antigen or protein, thereby causing rupture of the liposome or nano-container 191A, and modification of optical property of the content of liposome or nano-container 191A. Similarly, third type of liposomes or nano-containers 191C may be in contact with the hormone or third protein, thereby causing rupture of the liposome or nano-container 191C, and modification of optical property of the content of liposome or nano-container 191C. In contrast, the second type of liposomes or nano-containers 191B may not be in contact with the enzyme or second protein (e.g., if the enzyme is not present in the patient's body or GI tract), and the content of the second type of liposome or nano-container 191B may not be exposed or may not modify its optical property. Device 140 may acquire images including the first portion 192A, the second portion 192B, and the third portion 192C. The in-vivo images may indicate, for example, that the first and third antigens are detected and may be present in-vivo, whereas the second antigen is not detected and therefore may not be present in-vivo.

In some embodiments, reaction by the first type of liposomes or nano-containers 191A may result in a first modification of optical property (e.g., change of color from blue to yellow), whereas reaction by the by the second type of liposomes or nano-containers 191B may result in a second, different, modification of optical property (e.g., change of color from blue to green, or exposure of fluorescent substance).

In some embodiments, reaction by a first type of liposomes or nano-containers 191A may result in a first modification of optical property (e.g., change of color from blue to yellow); whereas reaction by the by a second type of liposomes or nano-containers 191B may result in a second, different, modification of non-optical properties, e.g., modification in magnetic field or conductivity, which may be detected by sensor 143.

In other embodiments, multiple reactions may result in similar, or even substantially identical, modifications of optical property, and may be differentiated or distinguished, for example, based on the location or the relative location of the portions. For example, a change of color from blue to yellow in the first portion 192A, may be distinguished from a change of color from blue to yellow in the third portion 192C, based on the location or the relative location of portions 192A and 192C on the band 192 as imaged by the in-vivo device 140. For example, a change of color from a first color to a second color at the location of the first portion 192A, may be used as indication that the first type of liposomes or nano-containers 191A reacted with a first type of antigen; whereas a change of color from the first color to the second color at the location of the first portion 192C, may be used as indication that the third type of liposomes or nano-containers 191C reacted with a third type of antigen.

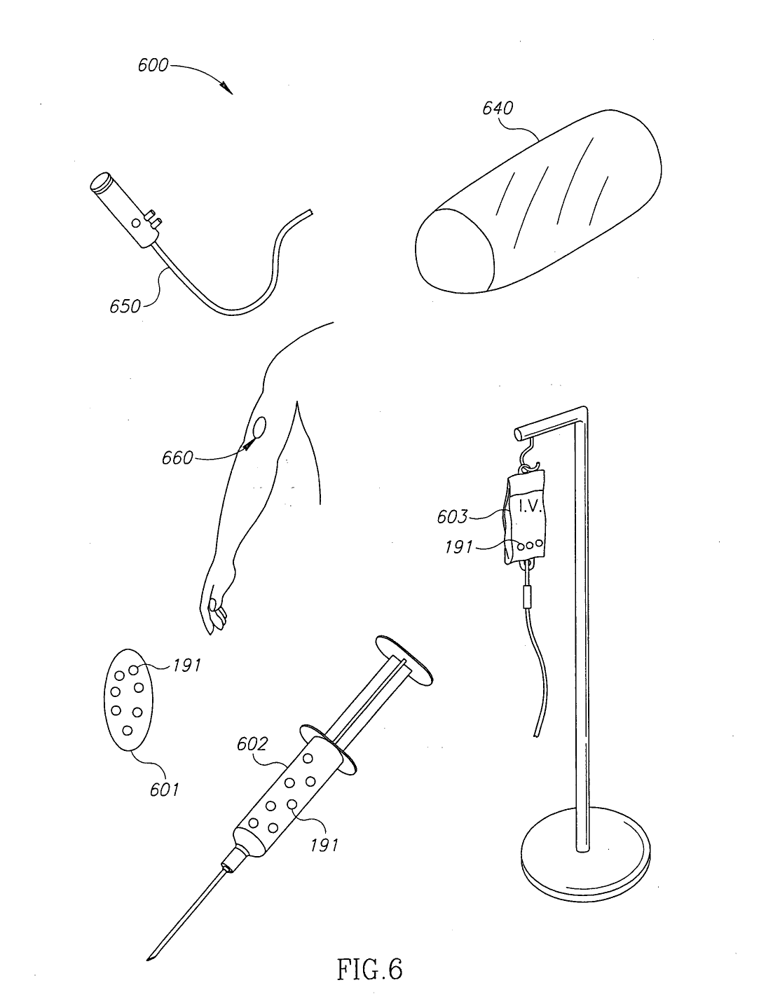

FIG. 6 schematically illustrates an in-vivo analysis system 600 in accordance with some embodiments of the invention. System 600 may include an in-vivo imaging device which may be autonomous or non-autonomous, for example, an autonomous in-vivo imaging device 640 and/or a non-autonomous in-vivo imaging device 650 and/or an implant 660.

The non-autonomous in-vivo imaging device 650 may include, for example, an endoscope or laparoscope. The autonomous in-vivo imaging device 640 may include, for example, a swallowable capsule, e.g., similar to in-vivo device 140 of FIG. 2, or a “PillCam”™ swallowable capsule produced by Given Imaging Ltd. The implant 660 may be, for example, an autonomous implant which may be implanted in-vivo. In some embodiments, device 640 and/or implant 660 may have in-vivo imaging capabilities as described herein, and/or may include other diagnostic capabilities, e.g., an Infra Red (IR) detector or an Ultra Violet (UV) detector. In some embodiments, device 640 and/or implant 660 need not include a substance carrier 190 (e.g., hydrogel) and/or need not include liposomes or nano-containers 191. System 600 may further include one or more delivery mechanisms able to deliver or insert liposomes or nano-containers 191 into a patient's body and/or into a certain part of organ of a patient's body, e.g., into a patient's blood stream, GI tract or other body part or body lumen. Such mechanisms may include, for example, a swallowable, digestible pill 601 containing liposomes or nano-containers 191; a syringe 602 or other injection mechanism (e.g., a hollow needle) able to inject liposomes or nano-containers 191; an intravenous (IV) system 603 able to inject liposomes or nano-containers 191, e.g., stored in a fluid form; or other suitable delivery mechanisms.

In some embodiments, for example, liposomes or nano-containers 191 may be inserted into a patient's body, e.g., using one or more of the delivery mechanisms 601-603. The in-vivo imaging device 640 or 650 may be inserted into the patient's body, for example, optionally after waiting a pre-defined time period during which the liposomes or nano-containers 191 may spread or may reach a certain body part or body organ. The liposomes or nano-containers 191 may react with a certain targeting agent like antigen or enzyme or hormone or any other protein in the patient's body, the reaction resulting in a rupture of the liposomes or nano-containers 191, and exposure and/or change of optical property of the signaling material stored in the liposomes or nano-containers 191. The in-vivo imaging device 640 or 650 or 660 may acquire images in-vivo; the images may include the exposed signaling agent and/or the signaling agent having a modified (e.g., non-original) color or optical property.

The acquired in-vivo images or signals may be transmitted or transferred to an external receiving or processing unit. The presence of a certain color or optical property of signaling material in the in-vivo images may indicate that the liposomes or nano-containers 191 reacted with a certain type of antigen. This may indicate, for example, the presence of that type of targeted agent within the patient's body.

In some embodiments, the in-vivo device 640 or 650 may be localized, e.g., using one or more localization methods, thereby allowing to determine the location or body part in which the reaction took place, e.g., the location or body part having the antigen, pathology, tumor, cancerous tumor, infection, polyp, or the like.

In some embodiments, optionally, the in-vivo device 640 or 650 or 660 may utilize Infra Red (IR) illumination and/or UV illumination and/or imaging technique, or other suitable techniques, for example, to illuminate and/or image the signaling agent, e.g., if the signaling agent includes a fluorescent material. For example, the in-vivo imaging device 640 or 650 or 660 may include an illumination unit (e.g., similar to illumination source 142 of FIG. 2) able to illuminate using a first wavelength of light to excite the florescence material; and an in-vivo imager (e.g., similar to in-vivo imager 146 of FIG. 2) or light detector (e.g., sensor 143) which may be sensitive to a second, different, wavelength of light, e.g., the wavelength of light emitted from the exposed signaling agent but not from the encapsulated signaling agent. This may allow, for example, IR or UV imaging of tissue or deep tissue marking. For example, liposomes or nano-containers 191 may be injected into the blood stream of the patient, and may contain IR sensitive compound that otherwise may not be injected to the blood (e.g., big insoluble molecule), which may react with a certain antigen within the patient's tissue, thereby causing accumulation of the signaling agent or a modification of optical properties of the signaling agent. The signaling material may be located within the body tissue, may not be noticeable or image-able using viewable light, yet may be imaged using Infra Red illumination and/or imaging.

In some embodiments, system 600 may utilize multiple types of liposomes or nano-containers 191. For example, multiple types of liposomes or nano-containers 191 may be administered to a patient's body, e.g., corresponding to multiple, respective, types of antigens or targeted agents. For example, the in-vivo imaging device 640 or 650 or 660 may acquire in-vivo images including a signaling agent having a first color, indicating the presence of a first targeting agent that reacted with a first type of liposomes or nano-containers 191; and optionally a second, different, color, indicating the presence of a second, different, targeting agent, that reacted with a second, different, type of liposomes or nano-containers 191.

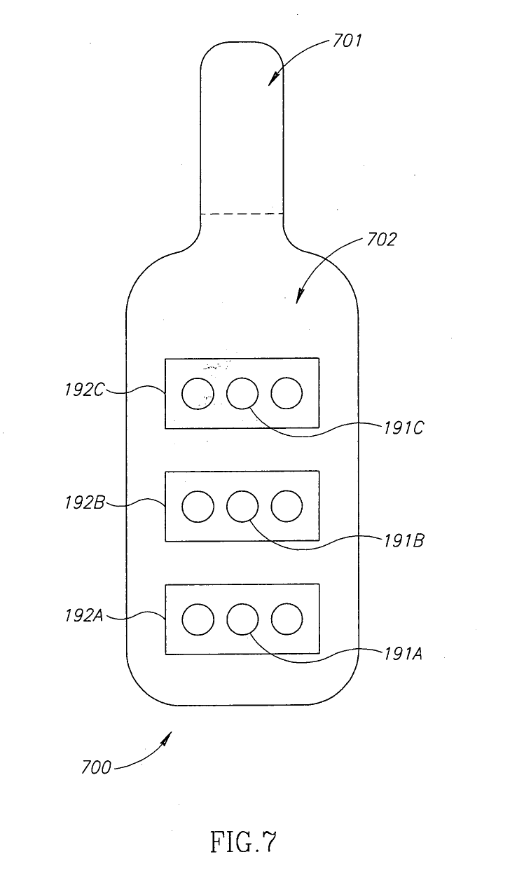

FIG. 7 schematically illustrates an analysis device 700 in accordance with some embodiments of the invention. Device 700 may be used, for example, in-vivo, ex-vivo, in-vitro, in a laboratory, in a clinic, in a hospital, at home, by a physician, by a patient, or the like.

Device 700 may optionally include a handle portion 701, for example, allowing a user to hold or grip the device 700; and a testing container or reaction surface containing lyophilized nanocontainers 702. Although a plane-shaped device 700 and a plane-shaped testing surface 702 are shown, other suitable shapes may be used, and device 700 and/or testing surface 702 may be non-planar. For example, in some embodiments, device 700 may be cylinder shaped and/or may include silicon.

Testing container 702 may include one or more testing portions, for example, testing containers or surfaces 192A, 192B and 192C, which may include, respectively, one or more types of liposomes or nano-containers, e.g., liposomes or nano-containers 191A, 191B and 191C.

For example, testing portion 192A may include a first type of liposomes or nano-containers 191A able to react with a first type of antigen; testing portion 192B may include a second type of liposomes or nano-containers 191B able to react with a second type of antigen; and testing portion 192C may include a third type of liposomes or nano-containers 191C able to react with a third type of antigen.

Testing surface 702, and/or testing portions 192A-C, may be in contact with a testing sample, for example, blood, plasma, urine, saliva, or the like. For example, testing surface 702 may be inserted in-vivo to collect a testing sample, and may then removed; or, testing surface 702 may remain ex-vivo, for example, the testing sample may be placed on testing surface 702, or testing surface 702 may be dipped within the testing sample. In some embodiments, medical testing samples may be used; in other embodiments, non-medical testing samples may be used, e.g., a water sample, a fluid sample, a food sample, an ecological sample, a biological sample, a chemical sample, or the like.

In some embodiments, the liposomes or nanocontainers 191 may be filled with a pH sensitive indicator. One or more types of antigens, which may be included in the testing sample, may react with one or more types of liposomes or nano-containers 191. For example, the first type of liposomes or nano-containers 191A may contain Methyl Orange in acidic solution thus having a red color. The first type of liposomes or nano-containers 191A may be in contact with a first antigen which may be present in the neutral plasma testing sample, thereby causing rupture of the liposome or nano-container 191A, the content of liposome or nano-container 191A will change color, e.g., to yellow. Similarly, the third type of liposomes or nano-containers 191C may contain Congo Red in a similar setting and thus may be blue. Once it is in contact with a third antigen which may be present in the same plasma testing sample, a rupture of the liposome or nano-container 191C may occur and the color of the liposome or nano-container 191C may change, e.g., to red. In contrast, the second type of liposomes or nano-containers 191B may contain Bromophenol Blue in a similar setting, and thus may be yellow; but if the second antigen is not present in the same plasma testing sample, the content of the second type of liposome or nano-container 191B may not be exposed to the natural pH plasma, and it may remain yellow and may not change color.

In some embodiments, marker(s) or signaling material(s) within the various types of liposomes or nano-containers 191 may be exposed, or may modify their optical properties (e.g., color, fluorescence, or the like), subsequent to rupture of the liposomes or nano-containers 191 due to their reaction with the corresponding antigen(s). This may result in, for example, an exposure or modification of optical property of the content of liposomes or nano-containers 191, e.g., signaling material(s) therein. The exposed or modified content may be viewable by a user, may be viewable using optical equipment. The optical properties of the exposed or modified content may indicate that certain liposomes or nano-containers 191 reacted with a certain, corresponding, antigen; thereby indicating that the testing sample contained that antigen.

In some embodiments utilizing device 700, reaction by the first type of liposomes or nano-containers 191A may result in a first modification of optical property (e.g., change of color from blue to yellow), whereas reaction by the by the second type of liposomes or nano-containers 191B may result in a second, different, modification of optical property (e.g., change of color from red to green, or exposure of fluorescent substance).

In some embodiments utilizing device 700, multiple reactions may result in similar, or even substantially identical, modifications of optical property, and may be differentiated or distinguished, for example, based on the location or the relative location of the portions 192. For example, a change of color from blue to yellow in the first portion 192A, may be distinguished from a change of color from blue to yellow in the third portion 192C, based on the location or the relative location of portions 192A and 192C on the testing surface 702. For example, a change of color from a first color to a second color at the location of the first portion 192A, may be used as indication that the first type of liposomes or nano-containers 191A reacted with a first type of antigen; whereas a change of color from the first color to the second color at the location of the first portion 192C, may be used as indication that the third type of liposomes or nano-containers 191C reacted with a third type of antigen.

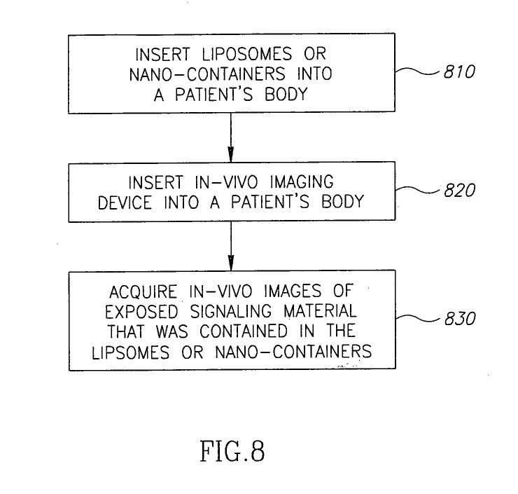

FIG. 8 is a flowchart depicting a method for in-vivo analysis in accordance with some embodiments of the invention. The method may be used, for example, in conjunction with system 600 of FIG. 6.

As indicated at box 810, the method may include, for example, inserting into a patient's body liposomes or nano-containers containing a signaling material, wherein the liposomes or nano-containers are able to react with a certain antigen, causing a rupture allowing exposure of the signaling material or modification of a color or other optical property of the signaling material. Inserting into a patient's body typically includes administering an effective amount of nano-containers.

The nano-containers of the present invention can be prepared and administered in a wide variety of oral and parenteral dosage forms. Thus, the nano-containers of the present invention can be administered by injection, that is, intravenously, intramuscularly, intracutaneously, subcutaneously, intraduodenally, or intraperitoneally. Also, the nano-containers of the present invention can be administered by inhalation, for example, intranasally. Additionally, the nano-containers of the present invention can be administered transdermally. To compositions useful in embodiments of the invention pharmaceutically acceptable carriers can be added. Solid form preparations can be use according to embodiments of the invention. Solid preparations may include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

The powders and tablets preferably contain from 5% or 10% to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations may be used according to embodiments of the invention. Liquid form preparations may include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

As indicated at box 820, the method may include, for example, inserting an in-vivo imaging device into the patient's body. This may be performed, for example, prior to or subsequent to the insertion of the liposomes or nano-containers into the patient's body.

As indicated at box 830, the method may include, for example, acquiring in-vivo images. For example, the in-vivo images may include the exposed or modified signaling material(s), thereby indicating the presence of certain antigen(s) associated with the liposomes or nano-containers that contained that signaling material(s). Other suitable operations or sets of operations may be used.

Devices, systems and methods in accordance with some embodiments of the invention may be used, for example, in conjunction with a device which may be inserted into a human body or swallowed by a person. However, embodiments of the invention are not limited in this regard, and may be used, for example, in conjunction with a device which may be inserted into, or swallowed by, a non-human body or an animal body.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for detecting the presence of a substance, the method comprising:
maintaining a signaling material at a first pH within a liposome;
contacting the liposome with a sample having a second pH such that if a substance is present in the sample the liposome will rupture thereby exposing the signaling material to the second pH; and
detecting a change in an optical property of the signaling material, said change caused by the exposure to the second pH.

2. The method according to claim 1 comprising detecting a change in color of the signaling material.

3. The method according to claim 1 comprising maintaining the signaling material at a basic pH within the liposome.

4. The method according to claim 1 wherein the second pH is the pH in a stomach.

5. The method of claim 1 comprising maintaining the signaling material within a liposome, said liposome adhered to a window of a swallowable imaging capsule.

6. The method according to claim 5 comprising imaging the change in the optical property.

7. The method according to claim 6 comprising wirelessly transmitting image data to a receiving system.

8. A method for detecting the presence of a substance, the method comprising:
maintaining a fluorescent material within a liposome;
contacting the liposome with a sample such that if a substance is present in the sample the liposome will rupture thereby exposing the fluorescent material; and
detecting a change in excitation property of the fluorescent material.
